(19) United States
(12) Patent Application Publication
(10) Pub. No.: US 2006/0231091 A1
(43) Pub. Date: Oct. 19, 2006

(54) Non-rebreathing oxygen face mask with nebulizer attachment

(76) Inventor: Melinda Camarillo, New Braunfels, TX (US)

Correspondence Address: EDWIN TARVER, 16830 Ventura Blvd., SUITE 360, Encino, CA, 91436, US

(21) Appl. No.: 11/109,101

(22) Filed: Apr. 18, 2005

(51) Int. Cl. A61M 11/00

(52) U.S. Cl. 128/200.21

(57) ABSTRACT

A non-rebreathing oxygen face mask for hospitals and other clinical settings comprising a face mask, a non-rebreather bag which serves as a reservoir for the aerosol treatment, a one-way valve connected to the bottom of the non-rebreather bag that serves as a nebulizer attachment source, a nebulizer containing medication in the form of a liquid, and plurality of tubes which are connected to the face mask and the nebulizer to supply oxygen and/or air from a gas source is disclosed in the present invention. The face mask adheres to the face of a patient with elastic straps and serves to deliver air, oxygen, and aerosolized medication to the patient and has vents to exhaust expelled air to the atmosphere. The non-rebreathing device will be more beneficial for patients who require a high concentration of oxygen to have nebulizer/respiratory treatment, without the need to remove the device.

### CROSS-REFERENCE TO RELATED APPPLICATION

None

### FEDERALLY SPONSORED RESEARCH

Not Applicable

### SEQUENCE LISTING OR PROGRAM

Not Applicable

### FIELD OF INVENTION

The present invention relates generally to artificial respiration devices, and more specifically to a medication administering non-rebreathing oxygen face mask with a nebulizer for use in hospitals and other clinical settings.

### BACKGROUND

Patients having respiratory problems are often administered pure oxygen through a non-rebreather mask. Such patients, in addition, will often require inhaled medications. Such medications are most frequently administered using a device commonly known as a “nebulizer”.

In the prior art, numerous attempts have been made to improve the function of oxygen masks used in patient care. These improvements have ranged from utilization improvements to patient comfort improvements. For example, U.S. Pat. No. 4,865,027 to Laanen et al., describes a reservoir bag connected in series to both a drug nebulizer and a mask having an inlet with a one-way valve. Meanwhile, U.S. Pat. No. 5,586,551 to Hilliard discloses a non-rebreather oxygen mask in communication with a nebulizer unit that separately delivers oxygen and an aerosolized medicant to the mask through a one-way valve.

Further, in U.S. Pat. No. 6,386,198 to Rugless, a multi-purpose oxygen face mask which functions as three different masks at once: regular, aerosol, and non-rebreather is disclosed. Additionally, U.S. Pub. No. 20030196664 to Jacobson describes an inhalation breathing apparatus that is used for administering a respirable fluid.

There are, however, a variety of problems associated with such nebulizer systems. One such problem is that they typically are complex and bulky. Respiration assemblies suffering from this deficiency will generally increase the discomfort of the patient. Moreover, such devices in the prior art are either structurally different than the embodiment of the present invention or to have nebulizer/respiratory treatment such non-rebreather device needs to be removed and hence not being beneficial to the patients.

Hence, the present invention is designed to overcome the shortcomings in the prior art and to provide an artificial respiration device and more specifically to provide a non-rebreathing oxygen face mask with nebulizer that can be used in all hospitals and/or clinical settings.

It is an object of the invention to provide a device that delivers a high concentration of oxygen useful for patients in need of artificial respiration, without removal of said device.

A further object of the invention is to provide a non-rebreathing mask with a one-way valve connected to a non-breather bag that will serve as a nebulizer attachment source.

Still another object of the invention is to provide a device with a non-rebreather bag wherein said bag will serve as a reservoir for the aerosol treatment, so that there is less wastage of the medication given by the nebulizer and hence, being more beneficial for the patient.

These and other objects will become apparent from the accompanying drawings and the description, which follows.

### SUMMARY

The present invention relates generally to an artificial respiration device and more specifically to a non-rebreathing oxygen face mask with a nebulizer that can be used in hospitals or other clinical settings. The non-rebreather mask has an air or oxygen source tube connected at the junction of a breathing mask and a non-rebreather bag. A one-way valve is incorporated into the bottom end of the non-rebreather bag. A nebulizer that accommodates an air or oxygen tube can be attached to the one-way valve.

When a patient requires an administration of aerosol medication, the nebulizer can be detached and filled with medicine. During this process, the patient breathes through the face mask, and is supplied air or oxygen through the tube connected to the top of the non-rebreather bag. After liquid medication has been incorporated into the nebulizer, a second air or oxygen tube is connected to the nebulizer, and the nebulizer is connected to the one-way valve. At this point, air or oxygen is administered through the nebulizer, and removed from the upper tube. In this manner, a patient can be administered liquid aerosolized medication through the non-rebreather mask without having to remove the mask.

### BRIEF DESCRIPTION OF THE FIGURES

FIG. 1 is a front view of a non-rebreather oxygen face mask with nebulizer in accordance with the present invention.

FIG. 2 is a side elevational view of a nebulizer to be attached to a one-way valve of a non-rebreather oxygen face mask of the present invention.

FIG. 3 is a side elevational view of the junction between a face mask and non-rebreather bag with an incorporated air or oxygen tube of the present invention.

### FIGURES—REFERENCE NUMERALS

10 . . . Non-rebreather Device
11 . . . Face Mask
12 . . . Non-rebreather Bag
13 . . . One-Way Valve with Cap
14 . . . Nebulizer Cup
15 . . . Nebulizer Dome with One-Way Valve Attachment
16 . . . Inlet for Nebulizer Bottle
17A . . . Upper Tube attached to Face Mask
17B . . . Lower Tube attached to Nebulizer
18 . . . Oxygen Inlet

### DESCRIPTION

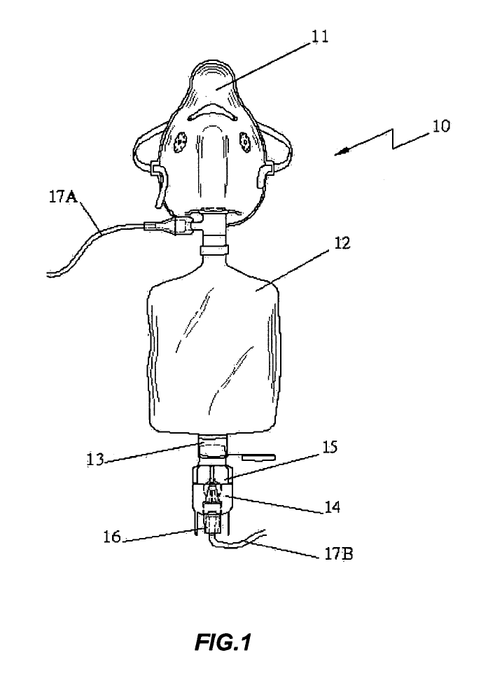

Referring to the drawings, the preferred embodiment of the non-rebreathing oxygen facemask with nebulizer device of the present invention is illustrated and generally indicated as 10 in FIG. 1. The non-rebreathing oxygen face mask 10 comprises of a face mask 11, which is generally shaped to follow the contour of the face around the nose and mouth. It adheres to the face of a patient with elastic straps and serves to deliver air, oxygen and aerosolized medication to the patient, and has vents to exhaust expelled air to the atmosphere.

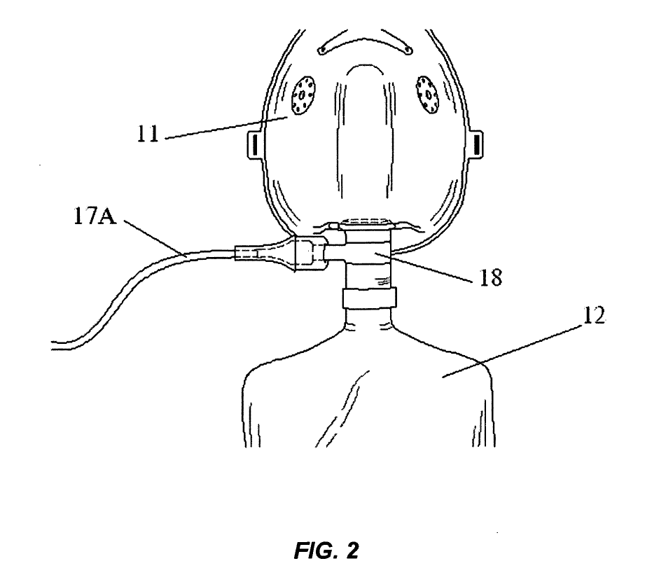

A modified non-rebreather bag 12 attaches to the face mask 11. As shown in FIG. 2, at the junction of the non-rebreather bag 12 and face mask 11, an oxygen inlet 18 is provided to accommodate an oxygen tube 17A. This upper tube 17A delivers oxygen to the patient when no medication is being administered.

The bottom of the non-rebreather bag 12 incorporates a capped one-way valve 13 at its lower end. The one-way valve 13 at the bottom of the non-rebreather bag serves as an attachment means for a nebulizer cup 14 and dome 15 assembly. The non-rebreather bag 12 serves as a reservoir for aerosolized medication emitted from the nebulizer 14 through the one-way valve 13.

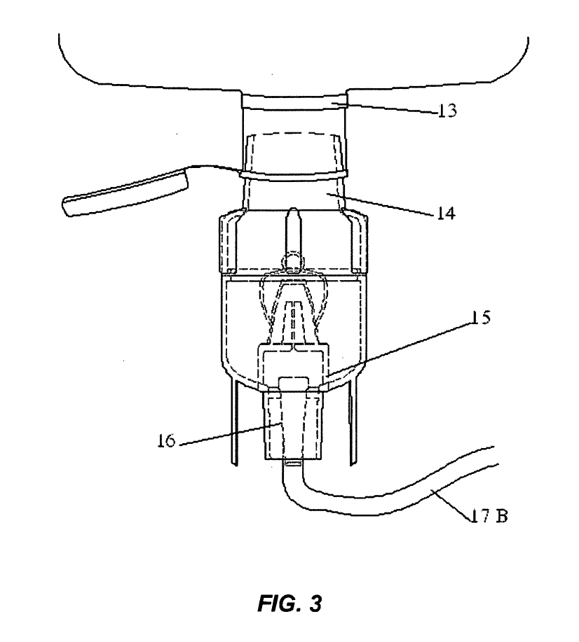

As shown in FIG. 3, the nebulizer cup 14 can accommodate liquid medication and has an attachment means at its base for an air or oxygen supply line. The nebulizer cap 15 closes over the cup 14, and attaches to the uncapped one-way valve 13 at the bottom of the non-rebreather bag 12.

In order to administer medication, an air or oxygen supply is administered through the lower tube 17b, nebulizer 14 and one-way valve 13, filling the rebreather bag 12. When the administration is complete, a secondary oxygen flow is administered from the upper oxygen tube. The nebulizer can be disconnected from the one-way valve 13 and the one-way valve 13 capped to prevent outside air from entering the non-rebreather bag 12. The non-rebreather bag 12 serves as a reservoir for the aerosol treatment emitted from the nebulizer, ensuring that the patient receives all of the administered medication.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

While specific apparatus has been disclosed in the preceding description, it should be understood that these specifics have been given for the purpose of disclosing the principles of the present invention and that many variations thereof will become apparent to those who are versed in the art. Therefore, the scope of the present invention is to be determined by the appended claims.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. A respiratory device comprising: a face mask that delivers oxygen at a high concentration and/or medication to the patient and vents exhaled air to atmosphere, a non-rebreather bag which serves as a reservoir for the aerosol treatment, a one-way valve connected to the bottom of the non-rebreather bag that serves as a nebulizer attachment source, a nebulizer containing medication in the form of a liquid, and plurality of tubes which are connected to the face mask and the nebulizer to supply oxygen and/or air from a gas source.

2. The respiratory device of claim 1, wherein said face mask is shaped to follow the contour of the patient's face around the nose and mouth, and has openings in the nose region that are connected to both the non-rebreather bag and a tube carrying the main oxygen supply from the gas source.

3. The respiratory device of claim 1, wherein the non-rebreather bag can be modified to include the one-way valve.

4. The respiratory device of claim 1, wherein said one-way valve is open only when the nebulizer is attached and can be capped by a cap, thus converting the respiratory device functionally to a conventional non-rebreathing mask.

5. The respiratory device of claim 1, wherein said nebulizer further comprises of a nebulizer bottle which is a cup-shaped container and in which the liquid medication is placed.

6. The respiratory device of claim 5, wherein the nebulizer bottle has an inlet for air or oxygen supply tube which is connected to air or oxygen gas source.

7. The respiratory device of claim 1, wherein said nebulizer is removably attached to other end of the one-way valve and such an arrangement allows the nebulizer to be removed when administration of the inhaled medication is no longer required.

8. The respiratory device of claim 7, wherein a cap is threadably mounted on the other end of the one-way valve when said nebulizer is removed.

9. The respiratory device of claim 1, wherein the nebulizer fits securely in the one-way valve, and helps in easy removal of said nebulizer when no longer needed.
